(19) United States
(12) Patent Application Publication
(10) Pub. No.: US 2015/0268356 A1
(43) Pub. Date: Sep. 24, 2015

(54) SEMICONDUCTOR RADIATION DETECTOR AND NUCLEAR MEDICINE DIAGNOSIS DEVICE USING THAT DETECTOR

(71) Applicant: HITACHI ALOKA MEDICAL, LTD., Mitaka-shi (JP)

(72) Inventors: Shinya KOMINAMI, Mito (JP); Kumi MOTAI, Tokyo (JP); Keiji KOBASHI, Mito (JP)

(21) Appl. No.: 13/798,380

(22) Filed: Mar. 13, 2013

(30) Foreign Application Priority Data
May 16, 2012 (JP) 2012-112665

(51) Int. Cl. G01T 1/161; G01T 1/24

(52) U.S. Cl. CPC G01T 1/161; G01T 1/249

(57) ABSTRACT

The present invention provides a semiconductor radiation detector including a semiconductor crystal sandwiched between a cathode electrode and an anode electrode, and a nuclear medicine diagnosis device using the semiconductor radiation detector. The semiconductor crystal is composed of a single crystal of thallium bromide of which the concentration of lead taken as an impurity is less than 0.1 ppm.

### BACKGROUND OF THE INVENTION

1. Field of the Invention

The present invention relates to a semiconductor radiation detector and a nuclear medicine diagnosis device using the same.

2. Description of the Related Art

A nuclear medicine diagnosis device using a radiation detector that measures radiation such as γ-rays is becoming widespread in recent years. As typical nuclear medicine diagnosis devices, may be mentioned a gamma camera device, a single photon emission computed tomography imaging device (SPECT imaging device), a positron emission tomography imaging device (PET imaging device), etc. The needs of a radiation detector in homeland security are growing in a dirty bomb counter-terrorism dosimeter or the like using a radiation detector.

These radiation detectors have heretofore been those each obtained by a combination of a scintillator and a photomultiplier. Attention has, however, recently been focused on a technology using a semiconductor radiation detector composed of a semiconductor crystal such as cadmium telluride, cadmium zinc telluride, or gallium arsenide, thallium bromide.

Since the semiconductor radiation detector has a configuration in which an electric charge produced by an interaction between radiation and a semiconductor crystal is converted to an electric signal, it has various features such as the efficiency of conversion to the electric signal, which is good as compared with one using the scintillator, and the feasibility of its size reduction.

The semiconductor radiation detector includes a semiconductor crystal, a cathode electrode formed in one surface of the semiconductor crystal, and an anode electrode opposite to the cathode electrode with the semiconductor crystal interposed therebetween. By applying a dc high voltage between these cathode and anode electrodes, an electric charge produced when radiation such as X-rays or γ-rays enters into the semiconductor crystal is taken out as a signal from the cathode or anode electrode.

Here, of the semiconductor crystals, especially, the thallium bromide is large in linear attenuation coefficient based on a photoelectric effect as compared with other semiconductor crystals such as cadmium telluride, cadmium zinc telluride, or gallium arsenide. γ-ray sensitivity equivalent to other semiconductor crystals can be obtained by a thin crystal. Therefore, a semiconductor radiation detector composed of thallium bromide and a nuclear medicine diagnosis device using the same can be made smaller in size than other semiconductor radiation detectors and a nuclear medicine diagnosis device using the same.

Also the thallium bromide is cheaper than other semiconductor crystals such as cadmium telluride, cadmium zinc telluride, or gallium arsenide. Therefore, a semiconductor radiation detector composed of thallium bromide and a nuclear medicine diagnosis device using the same can be made lower in cost than other semiconductor radiation detectors and a nuclear medicine diagnosis device using the same.

In the semiconductor radiation detector using thallium bromide as the semiconductor crystal, a γ-ray energy spectrum of 5.9 keV with ⁵⁵Fe as a radiation source, and a γ-ray energy spectrum of 59.6 keV with ²⁴¹Am as a radiation source have been observed (refer to, for example, Nuclear Instruments and Methods in physics Research Section-A, Vol. 591(2008), p. 209-212 (hereinafter referred to as Non-Patent Document 1)). In Non-Patent Document 1, however, energy spectra of γ-rays with ⁵⁷Co as a radiation source and γ-rays with ¹³⁷Cs as a radiation source have not been observed.

There has been disclosed in FIG. 1 of Non-Patent Document 1 that the concentration of lead taken as an impurity contained in a thallium bromide crystal used in a radiation detector is 10² ng/g (i.e., 0.1 ppm).

### SUMMARY OF THE INVENTION

Meanwhile, ^{99m}Tc has been known as a typical one of radioactive nuclides used in radioactive pharmaceuticals for nuclear medicine inspection by the gamma camera device, the SPECT imaging device or the like of the nuclear medicine diagnosis devices. An energy of major γ-rays emitted from ^{99m}Tc is 141 keV. It is an essential condition that a radiation detector used in the gamma camera device and the SPECT imaging device detects γ-rays of 141 keV. Thus, to examine the performance of the radiation detector for the gamma camera device and the SPECT imaging device, ⁵⁷Co that mainly emits γ-rays of 122 keV close in energy to 141 keV is often used as a standard radiation source.

In the nuclear medicine inspection by the PET imaging device of the nuclear medicine diagnosis devices, it is an essential condition that a pair of γ-rays having an energy 511 keV emitted in a 180-degree opposite direction is detected upon disappearance of each positron emitted from the radioactive pharmaceuticals. Thus, to the performance of a radiation detector for the PET imaging device, a ¹³⁷Cs radiation source that mainly emits γ-rays of 662 keV close in energy to 511 keV is often used as a standard radiation source.

However, when the thallium-bromide semiconductor radiation detector is fabricated by a related art technology, it cannot measure even energy spectra of both of γ-rays of 122 keV emitted from the ⁵⁷Co radiation source and γ-rays of 662 keV emitted from the ¹³⁷Cs radiation source and could not be used as the radiation detector for the gamma camera device and the SPECT imaging device and for the PET imaging device.

The lead of 0.1 ppm has been contained as the impurity in the thallium bromide crystal used in the radiation detector described in Non-Patent Document 1. Lead is an element adjacent to thallium in the periodic table. Since lead and thallium are both metallic elements, their atomic radii are defined by metallic bonding radii. According to a document (Fundamentals in Chemical Handbook, Fifth Revision, Edited by The Chemical Society of Japan), however, the atomic radius (metallic bonding radius) of thallium is 0.170 nm, whereas the atomic radius (metallic bonding radius) of lead is 0.175 nm. Thus, when lead atoms are being taken in as impurities, a substitutional solid solution is liable to make by partial substitution of thallium atoms. Further, each of the thallium atoms is liable to be the valence I, whereas each of the lead atoms is liable to be the valence II. Therefore, a spot where each lead atom is substituted is liable to be a defect as a crystal. To allow the thallium bromide crystal as a semiconductor radiation detector and obtain a high energy resolution, there is a need to collect or acquire most of charge carriers produced by the passage of incident radiation. It is however considered that each charge carrier is trapped in the defect in the crystal obtained by substitution of the lead atoms and its trapping length becomes short, and γ-ray energy spectra of 122 keV and 662 keV cannot be measured.

An object of the present invention is to provide a semiconductor radiation detector capable of measuring γ-ray energy spectra of 122 keV and 662 keV, and a nuclear medicine diagnosis device using the semiconductor radiation detector.

In order to solve the above problems, the present invention provides a semiconductor radiation detector including a semiconductor crystal sandwiched between a cathode electrode and an anode electrode. The semiconductor crystal is composed of a single crystal of thallium bromide of which the concentration of lead taken as an impurity is less than 0.1 ppm. According to such a composition, since the concentration of lead atoms in a thallium bromide single crystal is low, the density of each defect in a crystal producible by substitution of lead atoms for thallium atoms becomes low and the trapping length of each charge carrier can be made long. Therefore, γ-ray energy spectra of 122 keV and 662 keV can be measured in high energy resolution as a radiation detector.

According to the present invention, there can be obtained a semiconductor radiation detector capable of measuring γ-ray energy spectra of 122 keV and 662 keV in high energy resolution, and a nuclear medicine diagnosis device using the semiconductor radiation detector.

### BRIEF DESCRIPTION OF THE DRAWINGS

FIGS. 1A and 1B are configuration diagrams of a semiconductor radiation detector according to one embodiment of the present invention;

FIG. 2 is a diagram for describing the concentration of lead taken as an impurity of a semiconductor crystal used in the semiconductor radiation detector shown in FIGS. 1A and 1B;

FIG. 3 is a circuit diagram showing a circuit configuration taken where radiation measurement is performed by using it in the semiconductor radiation detector shown in FIGS. 1A and 1B;

FIG. 4 is a diagram for describing time changes in bias voltage applied to the semiconductor radiation detector shown in FIGS. 1A and 1B;

FIGS. 5A and 5B are diagrams for describing γ-ray energy spectra measured using the semiconductor radiation detector shown in FIGS. 1A and 1B;

FIGS. 6A and 6B are diagrams for describing γ-ray energy spectra measured using the semiconductor radiation detector shown in FIGS. 1A and 1B;

FIG. 7 is a configuration diagram of a nuclear medicine diagnosis device that uses the semiconductor radiation detector shown in FIGS. 1A and 1B; and

FIG. 8 is a configuration diagram of a nuclear medicine diagnosis device that uses the semiconductor radiation detector shown in FIGS. 1A and 1B.

### DESCRIPTION OF THE PREFERRED EMBODIMENT

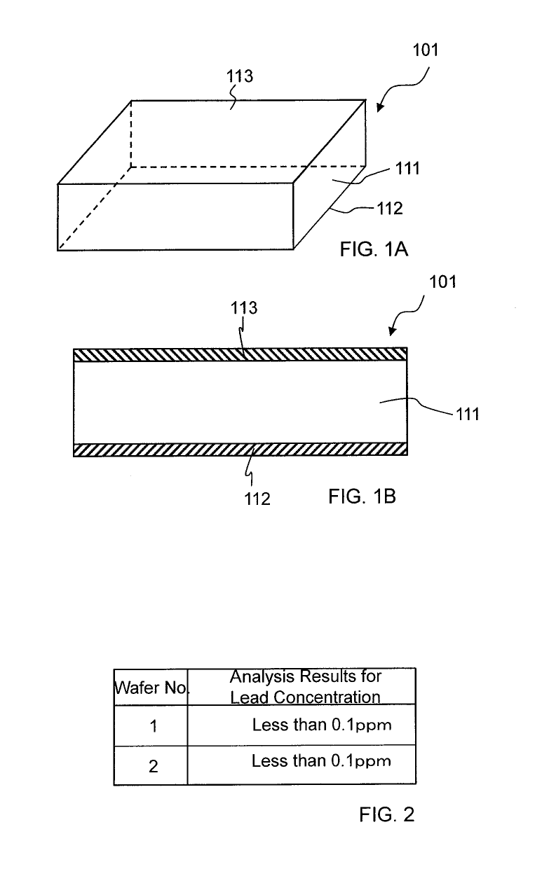

A description will hereinafter be made of configurations and operations of a semiconductor radiation detector according to one embodiment of the present invention and a nuclear medicine diagnosis device using the semiconductor radiation detector, using FIGS. 1A through 8.

The configuration of the semiconductor radiation detector will first be described using FIGS. 1A and 1B.

FIGS. 1A and 1B are configuration diagram of the semiconductor radiation detector according to the one embodiment of the present invention. FIG. 1A is a perspective view of the semiconductor radiation detector, and FIG. 1B is a sectional view thereof.

The semiconductor radiation detector (hereinafter called simply “detector”) 101 includes a sheet of semiconductor crystal 111 formed in a plate form, a first electrode 112 disposed at one surface (lower surface) of the semiconductor crystal 111, and a second electrode 113 disposed at the other surface (upper surface) thereof.

The semiconductor crystal 111 forms a region in which it interacts with radiation (y rays or the like) to generate electric charges and is formed by being cut from a single crystal of thallium bromide. The thallium bromide single crystal is grown by a single crystal growth apparatus after a commercially available 99.99% pure thallium bromide material has been subjected to purification processing. Incidentally, the thallium bromide material contains lead (Pb) as an impurity. As a method for the purification processing, may be mentioned a zone melting method, a vacuum evaporation method or the like. In the present embodiment, however, the process of purification was conducted aimed at reducing the concentration of lead taken as the impurity in the crystal. As a single crystal growth method, a vertical Bridgman method is used. The diameter of the crystal is about 3 inches. In the present embodiment, crystal growth was conducted twice using the same method to examine reproducibility of the process. As a result, two 3-inch single crystal ingots of Nos. 1 and 2 were obtained. The single crystal ingot is sliced by an annular saw slicing machine, followed by polishing, so that a 3-inch thallium bromide single crystal wafer having a thickness of 0.5 mm can be obtained.

A description will now be made of the concentration of the lead taken as the impurity of the semiconductor crystal used in the semiconductor radiation detector, using FIG. 2.

FIG. 2 is a diagram for describing the concentration of lead take as the impurity of the semiconductor crystal used in the semiconductor radiation detector.

FIG. 2 shows a result of glow discharge mass spectrometry (GDMS) conducted to examine the concentration of the lead taken as the impurity contained in each of the single crystal wafers Nos. 1 and 2 obtained from the two types of 3-inch single crystal ingots of Nos. 1 and 2. A limit of detection of the lead concentration by GDMS is 0.1 ppm, but no lead is detected from both wafers Nos. 1 and 2. The lead concentration is less than 0.1 ppm at both wafers.

The plate-like semiconductor crystal 111 shown in FIGS. 1A and 1B is obtained by dicing the single crystal wafer to, for example, a size of 5.1 mm×5.0 mm. Since the semiconductor crystal 111 fabricated from the wafer No. 1 and the semiconductor crystal 111 fabricated from the wafer No. 2 are both less than 0.1 ppm in lead concentration, they are reduced in lead concentration as compared with the related art thallium bromide crystal used in the radiation detector described in Non-Patent Document 1. Accordingly, a substitutional solid solution in which lead atoms substitute for some thallium atoms is less produced, and the defect density in the crystal is also reduced. Therefore, charge carries are trapped less frequently, and a long trapping length of each charge carrier can be obtained.

The first electrode 112 and the second electrode 113 are formed using either gold, platinum or palladium. The thickness of each electrode is assumed to be 50 nm, for example. The size of each of the first and second electrodes 112 and 113 is assumed to be 5.1 mm×5.0 mm, for example.

Incidentally, the sizes of the semiconductor crystal 111, the first electrode 112 and the second electrode 113 are shown by way of example. They are not limited to the respective sizes described above.

A process of fabricating the first electrode 112 and the second electrode 113 will next be explained.

First, gold, platinum or palladium is applied by 50 nm onto one surface (lower surface and size of 5.1 mm×5.0 mm) of a semiconductor crystal 111 composed of plate-like thallium bromide by an electron beam evaporation method to thereby form the first electrode 112.

Next, gold, platinum or palladium is applied by 50 nm onto the surface (upper surface and size of 5.1 mm×5.0 mm) of the semiconductor crystal 111, opposite to the surface of the formed first electrode 112 by the electron beam evaporation method to thereby form the second electrode 113.

A detector 101 is obtained through such a process.

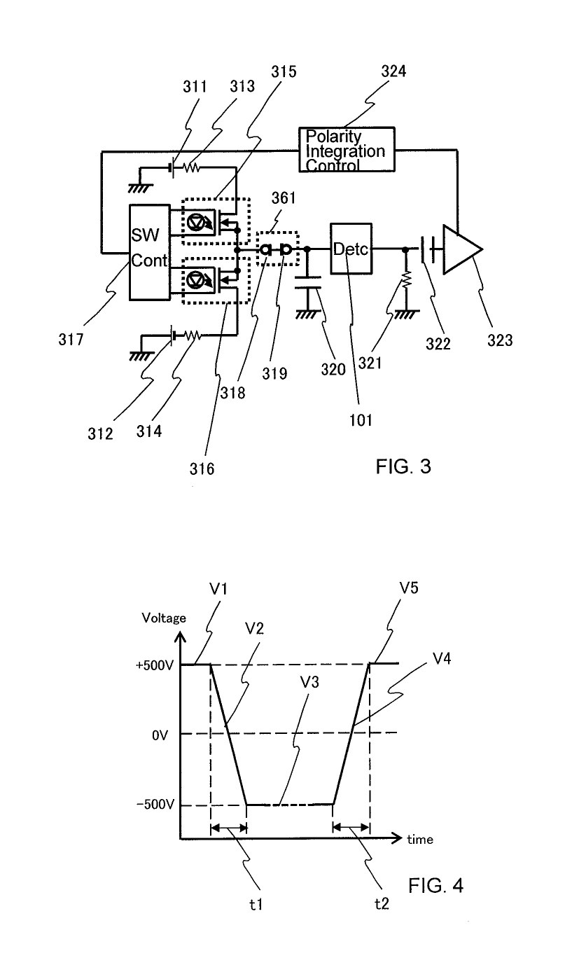

A circuit configuration taken where radiation measurement is conducted by using it in the semiconductor radiation detector according to the present embodiment will next be explained using FIG. 3.

FIG. 3 is a circuit diagram showing the circuit configuration taken where the radiation measurement is done by using it in the semiconductor radiation detector according to the one embodiment of the present invention.

In FIG. 3, a smoothing capacitor 320 which applies a voltage to the detector 101, a first DC power supply 311 which supplies a positive electric charge to one electrode of the smoothing capacitor 320, and a second DC power supply 312 which supplies a negative electric charge to the one electrode of the smoothing capacitor 320, are connected to the detector 101.

Further, a first constant current diode 318 made coincident in polarity of constant current characteristics so as to pass current through the one electrode of the smoothing capacitor 320 from the first DC power supply 311, and a second constant current diode 319 made coincident in polarity of constant current characteristics so as to pass current through the second DC power supply 312 from the one electrode of the smoothing capacitor 320 are connected between the first and second DC power supplies 311 and 312 and the detector 101.

Furthermore, a first photomos relay 315 is connected between the first DC power supply 311 and the one electrode of the smoothing capacitor 320. A second photomos relay 316 is connected between the second DC power supply 312 and the one electrode of the smoothing capacitor 320.

Still more, a protection resistor 313 is connected between the first DC power supply 311 and the first photomos relay 315. Also, a protection resistor 314 is connected between the second DC power supply 312 and the second photomos relay 316. The protection resistors 313 and 314 are resistors used for overcurrent prevention.

The closing and opening of each of the first photomos relay 315 and the second photomos relay 316 is controlled by a switching controller 317.

One electrode of a bleeder resistor 321 and that of a coupling capacitor 322 are connected to the output of the detector 101. An amplifier 323 which amplifies a signal of the detector 101 is connected to the other electrode of the coupling capacitor 322. Further, a polarity integration control device 324 that controls the opening and closing of the photomos relays 315 and 316 and the timing of polarity inversion of the amplifier 323, is connected to the switching controller 317 and the amplifier 323.

Other electrodes other than the negative electrode of the first DC power supply 311, the positive electrode of the second DC power supply 312, and the one electrode of the smoothing capacitor 320, and one electrode of the bleeder resistor 321 are connected to a ground wire.

Incidentally, the first constant current diode 318 and the second constant current diode 319 are connected in series with each other with being reversed in polarity of constant current characteristics to configure a constant current device 361. In this configuration, the general constant current diodes of the present situation used for the first constant current diode 318 and the second constant current diode 319 produce constant current characteristics with a structure in which source and gate electrodes of a field effect transistor (FET) are short-circuited. Therefore, when a reverse voltage is applied thereto, a p-n junction formed in the field effect transistor is biased in the forward direction, so that a large current flows. That is, the current characteristics of the constant current diodes have polarity. Thus, in the first constant current diode 318 and the second constant current diode 319, constant current characteristics with no difference in polarity are obtained by connecting the first and second constant current diodes 318 and 319 in series with each other with being reversed in polarity of their constant current characteristics.

When radiation such as γ rays is measured, a bias voltage for charge collection is applied between the first and second electrodes 112 and 113 of the detector 101 by the first DC power supply 311 or the second DC power supply 312 and the smoothing capacitor 320 (for example, +500 V or −500 V).

Here, since the semiconductor crystal 111 that is a component of the detector 101 is composed of thallium bromide, when, for example, the bias voltage of +500 V is continuously applied to the detector 101 using the first DC power supply 311, degradation in radiation measurement performance due to polarization, i.e., charge polarization occurs in the semiconductor crystal 111, and an energy resolution of γ-rays is degraded.

In order to prevent the polarization, the polarity of the bias voltage applied to the detector 101 needs to be periodically reversed. That is, it is necessary to invert the polarity from, for example, +500 V to −500 V and −500 V to +500 V. The cycle of its inversion is about 5 minutes.

A description will first be made of the case where the bias voltage of +500 V is applied to the detector 101. Since noise occurs when the voltage of +500 V is directly applied from the first DC power supply 311 to the detector 101, the voltage is applied to the detector 101 using the smoothing capacitor 320.

When the positive bias voltage is applied to the detector 101, the switching controller 317 closes the first photomos relay 315 and opens the second photomos relay 316.

The smoothing capacitor 320 is charged through the constant current device 361, so that the voltage of the smoothing capacitor 320 reaches +500 V. With its action, the bias voltage applied to the detector 101 also becomes +500 V. When the bias voltage of −500 V is applied to the detector 101 in reverse, a negative DC bias voltage is supplied by the second DC power supply 312.

When the negative bias voltage is applied to the detector 101, the switching controller 317 opens the first photomos relay 315 and closes the second photomos relay 316. The smoothing capacitor 320 is charged through the constant current device 361, so that the voltage of the smoothing capacitor 320 becomes −500 V. A positive or negative electric charge is accumulated at the one electrode of the smoothing capacitor 320 to thereby positively or negatively reverse the bias voltage applied to the detector 101.

The polarity integration control device 324 transmits a command signal indicative of each of “positive bias”, “negative bias”, “bias inversion from positive to negative” and “bias inversion from negative to positive” to the switching controller 317 and the amplifier 323, based on time information on polarity inversion for every 5 minutes. The switching controller 317 opens and closes the photomos relays 315 and 316, based on the command signal.

A description will now be made of time changes in bias voltage applied to the semiconductor radiation detector according to the present embodiment, using FIG. 4.

FIG. 4 is a diagram for describing the time changes in bias voltage applied to the semiconductor radiation detector according to the one embodiment of the present invention.

In the present embodiment, the bias voltage applied to the detector 101 is first a voltage V1 (+500 V) but changes to a voltage V3 (−500 V) due to the periodic inversion of the bias voltage. Five minutes afterwards, the bias voltage is reset to a voltage V5 (+500 V).

When the bias voltage is reversed, time changes in voltages V2 and V4 midway through its reversal become linear gradients. This is the advantage of the constant current device 361. While the bias voltage is being reversed, the absolute value of the bias voltage becomes insufficient as for charge collection so that a γ-ray detection signal cannot be taken out fully. On the other hand, however, discontinuous times for measurement (times t1 and t2 during which the voltages V2 and V4 are applied) are both 0.3 seconds. Although the discontinuous times of 0.3 seconds occur during a measurement of 5 minutes, when the semiconductor radiation detector is applied to the nuclear medicine diagnosis device or homeland security, they are sufficient short times and hence no problems arise.

When γ-rays enter into the detector 101 to which the bias voltage is applied, interactions occur between the semiconductor crystal 111 that configures the detector 101 and the incident γ-rays, so that an electric charge such as an electron and a positive hole is generated.

The generated electric charge is outputted from the detector 101 as a γ-ray detection signal. The γ-ray detection signal is inputted to the amplifier 323 via the coupling capacitor 322. The bleeder resistor 321 prevents electric charges from continuing to be accumulated in the coupling capacitor 322 and serves so as not to excessively increase the output voltage of the detector 101. The amplifier 323 serves to convert the γ-ray detection signal which is a small electric charge to its corresponding voltage and amplify the voltage.

The γ-ray detection signal amplified by the amplifier 323 is converted to a digital signal by a subsequent stage analog/digital converter (not shown), which in turn is counted by a data processing device (not shown) for every energy of γ-rays.

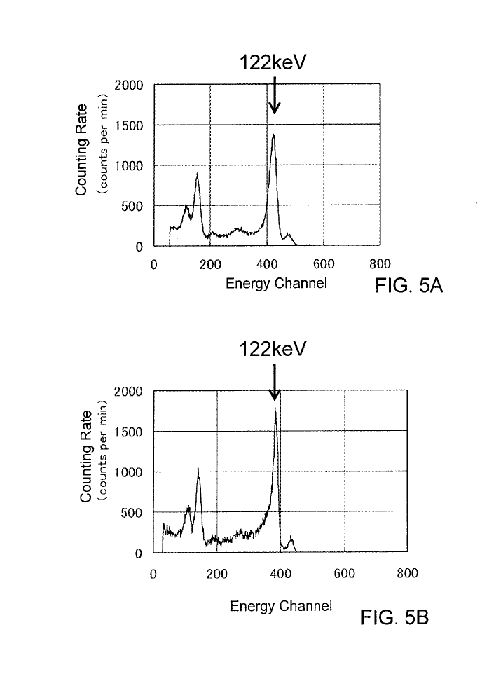

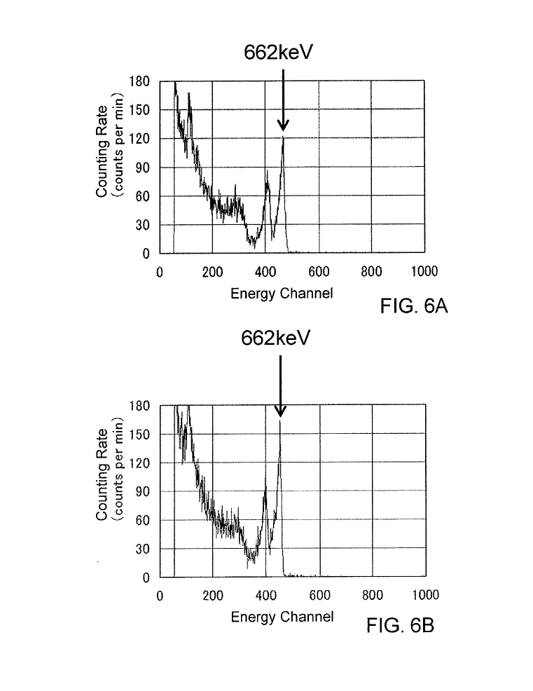

A description will next be made of γ-ray energy spectra measured using the semiconductor radiation detector according to the present embodiment using FIGS. 5A to 6B.

FIGS. 5A to 6B are diagrams for describing the γ-ray energy spectra measured using the semiconductor radiation detector according to the one embodiment of the present invention.

A description will first be made of γ-ray energy spectra of a ⁵⁷Co radiation source measured using the semiconductor radiation detector 101 of the present embodiment, using FIGS. 5A and 5B. FIG. 5A shows a result of measurement where the detector 101 is fabricated using the semiconductor crystal 111 cut out from the above wafer No. 1. FIG. 5B shows a result of measurement where the detector 101 is fabricated using the semiconductor crystal 111 cut out from the wafer No. 2.

In FIGS. 5A and 5B, the horizontal axis indicates a channel number of an energy channel. γ-rays of various energies are assigned to the energy channels of the respective numbers in correspondence with the respective channels according to the energies. In FIG. 5A, for example, a γ-ray energy of approximately 122 keV is assigned to an energy channel approximately in the neighborhood of a 420 channel. The vertical axis indicates a counting rate (counts per min) of γ-rays at the respective energy channels.

In FIG. 5A, a peak appears in the counting rate at the energy channel corresponding to the nearly 122 keV. An energy resolution at such a peak is represented as follows:

Energy resolution=(number of channels at half value width of peak)/(number of channels directly under peak)

In FIG. 5A, the energy resolution of 122 keV is approximately 8%. In FIG. 5B, the energy resolution of 122 keV is approximately 5%.

As described above, although a slight difference occurs between the energy resolutions where the detector 101 of the present embodiment shown in FIGS. 1A and 1B is configured using the semiconductor crystal 111 cut out from the wafer No. 1 and where the detector 101 is configured using the semiconductor crystal 111 cut out from the wafer No. 2, reproducibility is good and an energy spectrum of 122 keV is obtained together in both cases.

A description will next be made of γ-ray energy spectra of a ¹³⁷Cs radiation source measured using the semiconductor radiation detector 101 of the present embodiment, using FIGS. 6A and 6B. FIG. 6A shows a result of measurement where the detector 101 is fabricated using the semiconductor crystal 111 cut out from the wafer No. 1. FIG. 6B shows a result of measurement where the detector 101 is fabricated using the semiconductor crystal 111 cut out from the wafer No. 2. In FIGS. 6A and 6B, the horizontal axis indicates a channel number of an energy channel. The vertical axis indicates a counting rate (counts per min) of γ-rays at the respective energy channels.

In FIG. 6A, the energy resolution of 662 keV is approximately 5%. In FIG. 6B, the energy resolution of 662 keV is approximately 4%.

As described above, although a slight difference occurs between the energy resolutions where the detector 101 of the present embodiment shown in FIGS. 1A and 1B is configured using the semiconductor crystal 111 cut out from the wafer No. 1 and where the detector 101 is configured using the semiconductor crystal 111 cut out from the wafer No. 2, reproducibility is good and an energy spectrum of 662 keV is obtained together in both cases.

Thus, the detector 101 of the present embodiment is greatly improved in terms of the performance of radiation measurement at 122 keV and 662 keV as compared with the case where the detector is configured using the conventional thallium bromide crystal described in Non-Patent Document 1 as the semiconductor crystal. This is because in the detector 101 of the present embodiment, the semiconductor crystal 111 is composed of the single crystal of thallium bromide of which the lead concentration is less than 0.1 ppm.

With the use of the single crystal of thallium bromide of which the lead concentration is less than 0.1 ppm, as the semiconductor crystal, the density of defects in a crystal producible by substituting lead atoms for thallium atoms becomes small since the concentration of lead atoms in the thallium bromide single crystal is low, so that the trapping length of each charge carrier can be made long. Therefore, as the radiation detector, γ-ray energy spectra of 122 keV and 662 keV can be measured with high energy resolution.

Here, the use of the single crystal of thallium bromide of which the lead concentration is less than 0.1 ppm, as the semiconductor crystal makes it also possible to use a single crystal of thallium bromide of which the lead concentration is not greater than a detection limit of lead at glow discharge mass spectrometry (GDMS). By using such a semiconductor crystal, γ-ray energy spectra of 122 keV and 662 keV can be measured with high energy resolution as the radiation detector.

Using the single crystal of thallium bromide of which the lead concentration is less than 0.1 ppm, as the semiconductor crystal also enables a single crystal of thallium bromide of which the lead concentration is 0.0 ppm to be used as the semiconductor crystal. Here, the lead concentration being 0.0 ppm means that numerals of digits not greater than two significant digits may be any one. Lead concentrations not greater than, for example, 0.099 ppm, 0.09 ppm, 0.04 ppm and 0.01 ppm are contained. By using such a semiconductor crystal, γ-ray energy spectra of 122 keV and 662 keV can be measured with high energy resolution as the radiation detector.

Further, the use of the single crystal of thallium bromide of which the lead concentration is less than 0.1 ppm, as the semiconductor crystal makes it also possible to use a single crystal of thallium bromide uncontaining a substitutional solid solution of lead as the semiconductor crystal. This is because when the lead concentration is as low as less than 0.1 ppm, a substitutional solid solution is not formed by substitution of lead taken as an impurity for some of thallium atoms and no defect occurs, thereby resulting in that charge carriers are hard to be trapped and a trapping length of each charge carrier becomes longer. Therefore, the use of such a semiconductor crystal makes it possible to measure γ-ray energy spectra of 122 keV and 662 keV with high energy resolution as the radiation detector.

Furthermore, the use of the single crystal of thallium bromide of which the lead concentration is less than 0.1 ppm, as the semiconductor crystal makes it also possible to use a single crystal of thallium bromide free of such a defect that charge carriers are trapped, as the semiconductor crystal. This is because when the lead concentration is as low as less than 0.1 ppm, a substitutional solid solution is not formed by substitution of lead taken as an impurity for some of thallium atoms and no charge-carrier trapping defect occurs, thereby resulting in that charge carriers are hard to be trapped and a trapping length of each charge carrier becomes longer. Therefore, the use of such a semiconductor crystal makes it possible to measure γ-ray energy spectra of 122 keV and 662 keV with high energy resolution as the radiation detector.

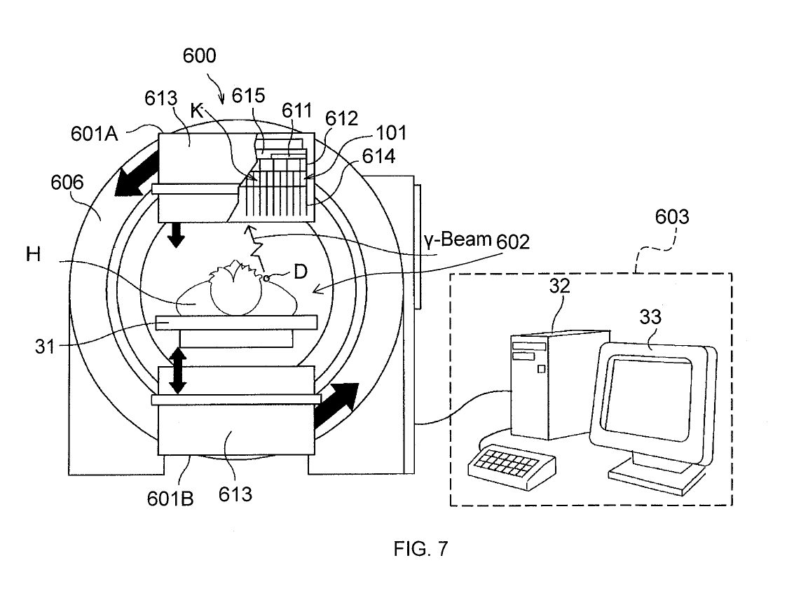

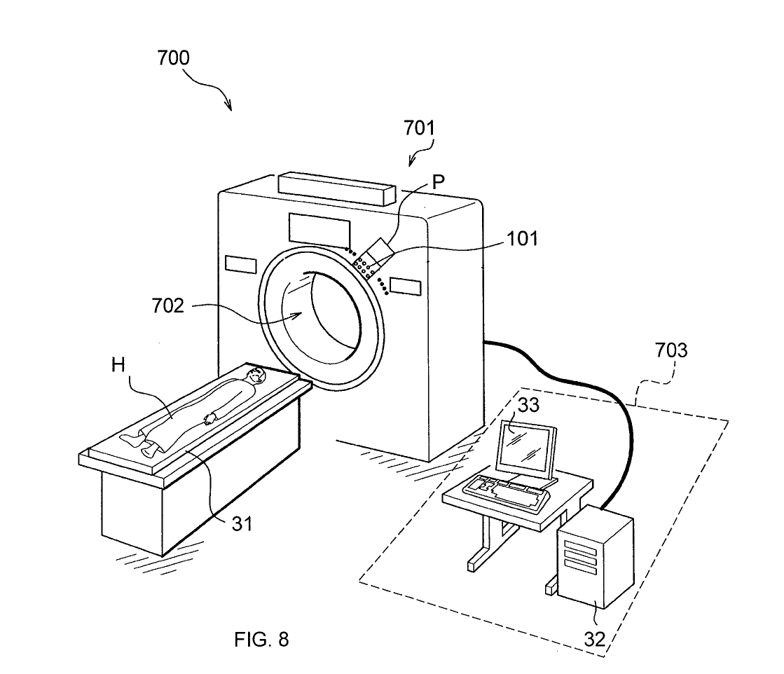

A configuration of a nuclear medicine diagnosis device using the semiconductor radiation detector according to the present embodiment will next be described using each of FIGS. 7 and 8.

FIGS. 7 and 8 are configurational diagrams of the nuclear medicine diagnosis devices using the semiconductor radiation detector according to the one embodiment of the present invention.

First, using FIG. 7, a description will first be made of the case where the detector 101 of the present embodiment is applied to a single photon emission computed tomography device (SPECT imaging device) as the nuclear medicine diagnosis device.

In FIG. 7, the SPECT imaging device 600 includes two radiation detection blocks 601A and 601B located above and below so as to surround a cylindrical measurement area 602 at its central part, a rotatable support base 606, a bed 31 and an image information creating device 603.

Here, the radiation detection block 601A placed on the upper side includes a plurality of radiation measurement units 611, a unit support member 615, and a lightproof electromagnetic shield 613. The radiation measurement unit 611 is equipped with a plurality of semiconductor radiation detectors 101, basal plates 612, and collimators 614. The radiation detection block 601B placed below also has a similar configuration. The image information creating device 603 is composed of a data processing device 32 and a display device 33.

The radiation detection blocks 601A and 601B are placed in positions shifted by 180 degrees from each other as viewed in a circumferential direction at the rotatable support base 606. Specifically, the unit support members 615 (only one shown in the drawing) of the radiation detection blocks 601A and 601B are attached to the rotatable support base 606 at positions spaced away from each other by 180 degrees in the circumferential direction. The radiation measurement units 611 including the basal plates 612 are detachably attached to the unit support members 615.

The detectors 101 are each disposed in multistage in areas K partitioned by the collimators 614 in a state of being attached to the basal plates 612. The collimators 614 are formed of a radiation shielding member (for example, lead, tungsten or the like) and form a number of radiation passages through which radiation (e.g., γ-rays) pass.

All the basal plates 612 and collimators 614 are disposed within the lightproof and electromagnetic shield 613 installed onto the rotatable support base 606. The lightproof electromagnetic shield 613 interrupts or cuts off the effect of electromagnetic waves other than γ-rays to the detectors 101 or the like.

In such a SPECT imaging device 600, the bed 31 with a subject H to be examined administered with radioactive pharmaceuticals being placed thereon is moved so that the subject H is moved between the pair of radiation detection blocks 601A and 601B. Then, the rotatable support base 606 is rotated so that the radiation detection blocks 601A and 601B are turned around the subject H to start the detection thereof.

When γ-rays are emitted from an accumulated region (e.g., an affected part) in the subject H with the radioactive pharmaceuticals accumulated therein, the emitted γ-rays enter the corresponding detector 101 through the radiation passage of each collimator 614. Then, the detector 101 outputs a γ-ray detection signal. The γ-ray detection signal is counted by the data processing device 32 for every energy of γ-rays, and information or the like thereof is displayed on the display device 33.

Incidentally, in FIG. 7, the radiation detection blocks 601A and 601B are rotated as indicated by thick arrows while being supported by the rotatable support base 606 to perform imaging and measurement of the subject H while changing the angle relative to the subject H. The radiation detection blocks 601A and 601B are movable upward and downward as indicated by thin arrows and hence capable of changing the distance to the subject H.

Each of the detectors 101 used in such a SPECT imaging device 600 is capable of measuring a γ-ray energy spectrum of 122 keV in high energy resolution while using thallium bromide as a semiconductor crystal. It is thus possible to provide a SPECT imaging device which is small-sized and low in cost and which is capable of imaging in high energy resolution, ^{99m}Tc that is a typical radioactive nuclide used in radiopharmaceuticals for nuclear medicine inspection and emits γ-rays of 141 keV.

Next, using FIG. 8, a description will be made of the case where the detector 101 of the present embodiment is applied to a PET imaging device 700 as a nuclear medicine diagnosis device.

The detector 101 of the present embodiment is not limited to the SPECT imaging device 600, but can be used even in a gamma camera device, a PET image device or the like as a nuclear medicine diagnosis device.

In FIG. 8, the positron emission tomography imaging device (PET imaging device) 700 is equipped with an imaging device 701 having a cylindrical measurement area 702 at its central part, a bed 31 which supports a subject H to be examined and is movable in its longitudinal direction, and an image information creating device 703. Incidentally, the image information creating device 703 is equipped with a data processing device 32 and a display device 33.

Each basal plate P equipped with a large number of the detectors 101 is arranged in the imaging device 700 so as to surround the measurement area 702.

In such a PET imaging device 700, there are provided a digital ASIC (Application Specific Integrated Circuit for digital circuit: not shown in the drawing) having a data process function, etc. A packet having energy values of γ-rays, times, a detection channel ID of each detector 101 is created. The so-created packet is input to the data processing device 32.

Upon inspection, γ-rays emitted from within the body of the subject H due to radioactive pharmaceuticals are detected by the detectors 101. That is, upon disappearance of each positron emitted from the radioactive pharmaceuticals for PET imaging, a pair of γ-rays is emitted in a 180-degree opposite direction and detected by discrete detection channels of the large number of detectors 101. Each of the detected γ-ray detection signals is input to the corresponding digital ASIC, where it is subjected to signal processing as described above. Information about the positions of the detection channels by which the γ-rays are detected, and information about the detected times of γ-rays are input to the data processing device 32.

Then, the data processing device 32 counts (simultaneously counts) a pair of γ-rays produced due to the disappearance of one positron as one and specifies the positions of the two detection channels by which the pair of γ-rays are detected, based on their position information. The data processing device 32 creates tomographic image information (image information) of the subject H at the accumulated position of radioactive pharmaceutical, i.e., the position of a tumor, using the counted values obtained by simultaneous counting and the position information of the detection channels. This tomographic image information is displayed on the display device 33.

Each of the detectors 101 used in such a PET imaging device 700 is capable of measuring a γ-ray energy spectrum of 662 keV in high energy resolution while using thallium bromide as a semiconductor crystal. It is thus possible to provide a PET imaging device which is small-sized and low in cost and which is capable of detecting in high energy resolution, γ-rays of 511 keV emitted from each positron produced from radioactive pharmaceuticals for PET inspection.

As described above, according to the present embodiment, γ-ray energy spectra of 122 keV and 662 keV can be measured in high energy resolution by a radiation detector while using thallium bromide as a semiconductor crystal that configures the radiation detector. Accordingly, it is possible to provide a semiconductor radiation detector which is small in size and low in cost and which is high in energy resolution, and a nuclear medicine diagnosis device having the semiconductor radiation detector.

Incidentally, the semiconductor radiation detector of the present invention and the nuclear medicine diagnosis device equipped therewith are capable of imaging radioactive pharmaceuticals in high energy resolution and achieving reductions in size and cost. Therefore, they make a contribution to their widespread use and are widely available and adopted in this field.

What is claimed is:

1. A semiconductor radiation detector comprising:
a semiconductor crystal sandwiched between a cathode electrode and an anode electrode,
wherein the semiconductor crystal is composed of a single crystal of thallium bromide of which the concentration of lead taken as an impurity is less than 0.1 ppm.

2. The semiconductor radiation detector according to claim 1, wherein the cathode and anode electrodes are each composed of metals more than at least one of gold, platinum and palladium.

3. A semiconductor radiation detector comprising:
a semiconductor crystal sandwiched between a cathode electrode and an anode electrode,
wherein the semiconductor crystal is composed of a single crystal of thallium bromide of which the concentration of lead taken as an impurity is not greater than a limit of detection of a lead concentration by glow discharge mass spectrometry (GDMS).

4. A semiconductor radiation detector comprising:
a semiconductor crystal sandwiched between a cathode electrode and an anode electrode,
wherein the semiconductor crystal is composed of a single crystal of thallium bromide of which the concentration of lead taken as an impurity is 0.0 ppm.

5. A nuclear medicine diagnosis device comprising:
basal plates to each of which a plurality of the semiconductor radiation detectors are attached, said basal plates surrounding a measurement area in which a bed supporting a subject to be examined thereon is inserted, and being disposed around the measurement area; and
an image information creating device which generates images using information obtained based on radiation detection signals output from the semiconductor radiation detectors of the basal plates,
wherein each of the semiconductor radiation detectors is a semiconductor radiation detector including a semiconductor crystal sandwiched between a cathode electrode and an anode electrode, wherein the semiconductor crystal is composed of a single crystal of thallium bromide of which the concentration of lead taken as an impurity is less than 0.1 ppm.

6. A nuclear medicine diagnosis device comprising:
basal plates to each of which a plurality of the semiconductor radiation detectors are attached, said basal plates surrounding a measurement area in which a bed supporting a subject to be examined thereon is inserted, and being disposed around the measurement area; and
an image information creating device which generates images using information obtained based on radiation detection signals output from the semiconductor radiation detectors of the basal plates,
wherein each of the semiconductor radiation detectors is a semiconductor radiation detector including a semiconductor crystal sandwiched between a cathode electrode and an anode electrode, wherein the semiconductor crystal is composed of a single crystal of thallium bromide of which the concentration of lead taken as an impurity is not greater than a limit of detection of a lead concentration by glow discharge mass spectrometry (GDMS).

7. A nuclear medicine diagnosis device comprising:
basal plates to each of which a plurality of the semiconductor radiation detectors are attached, said basal plates surrounding a measurement area in which a bed supporting a subject to be examined thereon is inserted, and being disposed around the measurement area; and
an image information creating device which generates images using information obtained based on radiation detection signals output from the semiconductor radiation detectors of the basal plates,
wherein each of the semiconductor radiation detectors is a semiconductor radiation detector including a semiconductor crystal sandwiched between a cathode electrode and an anode electrode, wherein the semiconductor crystal is composed of a single crystal of thallium bromide of which the concentration of lead taken as an impurity is 0.0 ppm.
